(19) United States
(12) Patent Application Publication
(10) Pub. No.: US 2003/0225432 A1
(43) Pub. Date: Dec. 4, 2003

(54) Soft tissue retraction device for an endoscopic instrument

(76) Inventors: Reginald C. Baptiste, Phoenix, AZ (US); Scott A. Sher, Scottsdale, AZ (US)

Correspondence Address: Charlene Jacobsen, MESCHKOW & GRESHAM, P.L.C., Suite 409, 5727 N. Seventh Street, Phoenix, AZ, 85014, US

(21) Appl. No.: 10/158,805

(22) Filed: May 31, 2002

(51) Int. Cl. A61M 29/00

(52) U.S. Cl. 606/191

(57) ABSTRACT

A soft tissue retraction device (20) is configured for use with an endoscopic instrument (26). The endoscopic instrument (26) includes a tubular body (28) having a proximal end (30) and a distal end (32) adapted for placement in a body cavity (38). The soft tissue retraction device (20) includes an expansion element (22) adapted to mount on the distal end (32) of the tubular body (28). An activation mechanism (24) is in communication with the expansion element (22) for adjusting the expansion element (22) from a collapsed configuration (21) to a deployed configuration (62). The deployed configuration (62) noninjuriously urges tissues (40) away from a target site (42) to form a working space (44) circumscribed by the expansion element (22) in which to surgically maneuver an endoscopic tool (36) of the endoscopic instrument (26). A method of using the soft tissue retraction device (20) when performing video-assisted thoracic surgery and when performing a laparoscopic procedure is also disclosed.

### TECHNICAL FIELD OF THE INVENTION

The present invention relates to the field of endoscopic instruments. More specifically, the present invention relates to methods and devices for creating a working space in a body cavity within which endoscopic tools can access a target site.

### BACKGROUND OF THE INVENTION

Many thoracic surgical procedures are performed for heart and lung disease, muscle and nerve disorders, ulcers and other serious illnesses. Although surgery may be the best, or only way to treat the disease, patients can sometimes face a long and difficult recovery because traditional “open” thoracic surgery is highly invasive. In an open thoracic surgery, known as a thoracotomy, surgeons must make a long incision through chest muscles and then cut and spread the patient's ribs to reach the diseased area. As a result, patients may spend up to a week in the hospital and up to four to six weeks of recovery at home.

The development of endoscopic video capability and instrumentation has resulted in the application of diagnostic and therapeutic thoracoscopy, also known as video-assisted thoracic surgery (VATS), to many disease processes encountered in thoracic medicine. VATS is a technique in which small diameter instruments such as cameras, graspers, forceps, retractors, dissectors, clamps, and so forth are inserted through small openings in the body to perform surgical procedures within the thoracic cavity. By utilizing a VATS procedure for exploring, diagnosing, and treating disease processes within the thoracic cavity, the pain, morbidity, and long recovery duration of more invasive procedures, such as the traditional large incision thoracotomy can often be avoided.

VATS procedures typically require double-lumen endotracheal intubation and single-lung ventilation. Working space in which to maneuver surgically in the chest is created by ventilating the opposite lung through the double-lumen endotracheal tube and allowing collapse of the affected lung after creation of a small intercostal incision. Collapse of the affected lung enables improved visibility of the lung, as well as virtually all the major structures in the chest cavity, to aid in exploration, treatment, and/or biopsy of a target site.

Unfortunately, some critically ill patients requiring high levels of airway pressure are unable to tolerate single lung ventilation. Consequently, these critically ill patients may not be candidates for a VATS procedure, and the traditional thoracotomy with its attendant pain, morbidity, and long recovery duration is still required. Another contraindication to a VATS procedure is the inability of the patient to tolerate a general anesthetic with single-lung ventilation. This situation can occur with mechanically ventilated patients in severe respiratory failure. Other patients for which VATS is virtually impossible are those who have undergone pneumonectomy and thus do not have an opposite lung to ventilate.

Unfortunately, intubation, with dual- or single-lung ventilation, and general anesthesia have the potential to cause a great number of side effects and complications. Minor side effects, causing pain and discomfort, include sore throat and damage to teeth (caused by the endotracheal tube), drowsiness, nausea and vomiting, headache, dizziness, and vision problems. Serious complications that can arise through the use of general anesthesia include stroke, heart attack, brain damage, and death.

Thus, what is needed is a device and method for creating a working space in the thoracic cavity for performing a (VATS) procedure in the presence of an inflated lung, without the necessity of utilizing intubation techniques and general anesthesia.

### SUMMARY OF THE INVENTION

Accordingly, it is an advantage of the present invention that a soft tissue retraction device for an endoscopic instrument is provided that noninjuriously urges tissues within a body cavity away from a target site.

It is another advantage of the present invention that a soft tissue retraction device is provided that can readily mount to a variety of existing and upcoming endoscopic instruments.

Yet another advantage of the present invention is that a method is provided for performing a video-assisted thoracic surgery in the presence of inflated lung tissue using an endoscopic instrument with a soft tissue retraction device.

The above and other advantages of the present invention are carried out in one form by a soft tissue retraction device for an endoscopic instrument used to access a body cavity, the endoscopic instrument including a tubular body having a proximal end and a distal end, the distal end being adapted for placement in the body cavity. The soft tissue retraction device includes an expansion element adapted to mount on the distal end of the tubular body and an activation mechanism in communication with the expansion element for adjusting the expansion element from a collapsed configuration to a deployed configuration. The deployed configuration of the expansion element noninjuriously urges tissues within the body cavity away from a target site to form a working space circumscribed by the expansion element in which to surgically maneuver the endoscopic instrument.

The above and other advantages of the present invention are carried out in another form by a method of performing video-assisted thoracic surgery (VATS) at a target site in a thoracic cavity of a body in the presence of inflated lung tissue. The method calls for creating an incision through an intercostal space of the body into the thoracic cavity. An endoscopic instrument, with a soft tissue retraction device mounted thereon, is inserted through the incision into the thoracic cavity. The endoscopic instrument includes a tubular body having a proximal end and a distal end, the distal end being adapted for placement in the body cavity, and the distal end having an endoscopic tool extending therefrom. The soft tissue retraction device includes an expansion element mounted on the distal end of the tubular body and an activation mechanism in communication with the expansion element for adjusting the expansion element from a collapsed configuration to a deployed configuration. The inserting operation is performed with the expansion element in the collapsed configuration. The method further calls for adjusting the expansion element to the deployed configuration via the activation mechanism to noninjuriously urge the inflated lung tissue within the thoracic cavity away from a target site to form a working space circumscribed by the expansion element. The endoscopic tool is maneuvered within the working space to access the target site.

### BRIEF DESCRIPTION OF THE DRAWINGS

A more complete understanding of the present invention may be derived by referring to the detailed description and claims when considered in connection with the Figures, wherein like reference numbers refer to similar items throughout the Figures, and:

FIG. 1 shows a perspective view of a soft tissue retraction device in accordance with a preferred embodiment of the present invention;

FIG. 2 shows a perspective view of the soft tissue retraction device of FIG. 1 in a collapsed configuration;

FIG. 3 shows an illustrative perspective view of the soft tissue retraction device of FIG. 1 in use;

FIG. 4 shows a perspective view a soft tissue retraction device mounted on an endoscopic instrument in accordance with an alternative embodiment of the present invention;

FIG. 5 shows a perspective view of the soft tissue retraction device of FIG. 4 in a collapsed configuration;

FIG. 6 shows a cutaway perspective view of the soft tissue retraction device of FIG. 4 in use;

FIG. 7 shows a perspective view of the soft tissue retraction device of FIG. 4 mounted downstream from a moveable joint of a tubular body of an endoscopic instrument;

FIG. 8 shows a cutaway perspective view of another alternative soft tissue retraction device in use;

FIG. 9 shows a perspective view of another alternative soft tissue retraction device;

FIG. 10 shows an illustrative perspective view of the soft tissue retraction device of FIG. 9 in use within the abdominal cavity of a patient;

FIG. 11 shows a perspective view of the soft tissue retraction device of FIG. 4 mounted on a trocar; and

FIG. 12 shows an illustrative perspective view of the soft tissue retraction device of FIG. 4 mounted on a trocar and in use within the abdominal cavity of a patient.

### DETAILED DESCRIPTION OF THE PREFERRED EMBODIMENTS

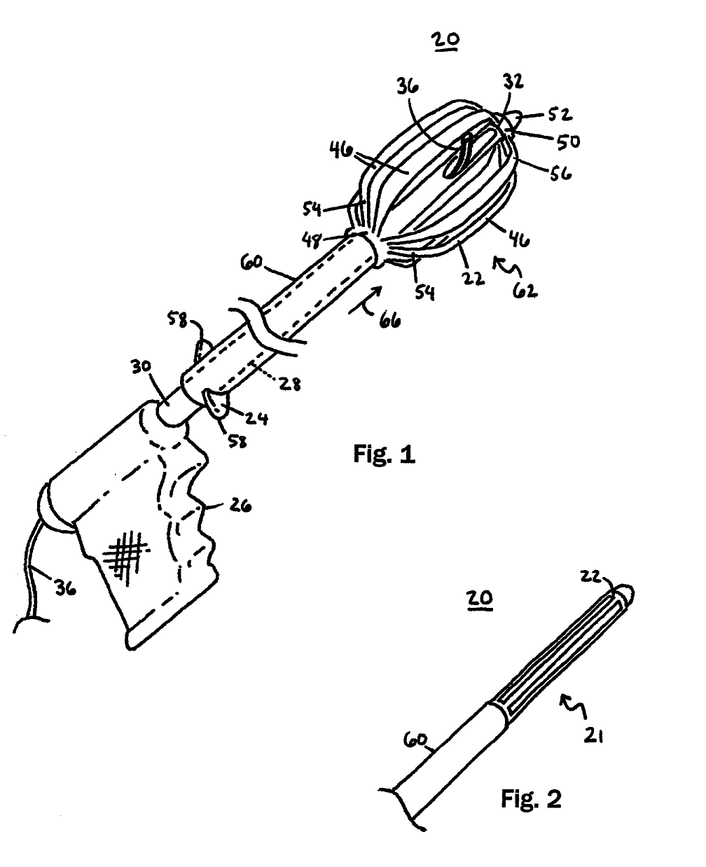

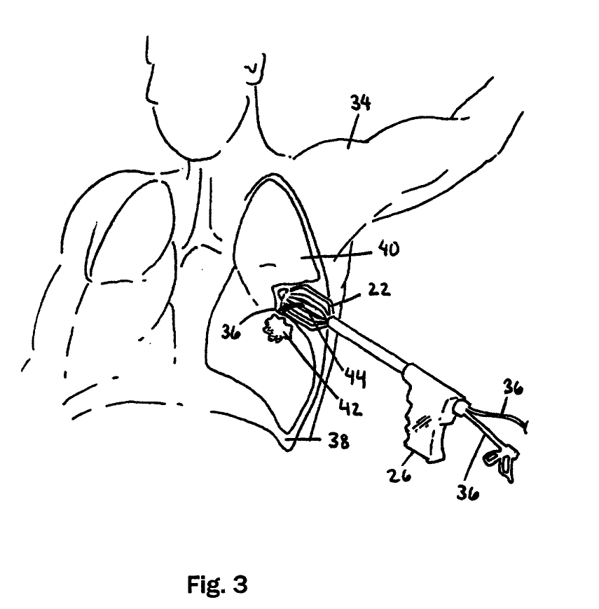

Referring to FIGS. 1-3, FIG. 1 shows a perspective view of a soft tissue retraction device 20 in accordance with a preferred embodiment of the present invention. FIG. 2 shows a perspective view of soft tissue retraction device 20 in a collapsed configuration 21, and FIG. 3 shows an illustrative perspective view of soft tissue retraction device 20 in use. Soft tissue retraction device 20 includes an expansion element 22 and an activation mechanism 24 in communication with expansion element 22.

Soft tissue retraction device 20 is configured for use with an endoscopic instrument 26. Endoscopic instrument 26 includes a tubular body 28 having a proximal end 30 and a distal end 32 adapted for placement in a body cavity of a patient 34. Endoscopic instrument 26 may include a number of endoscopic tools 36, best shown in FIG. 1, directed through one or more passages (not shown) within tubular body 28 from proximal end 30 and exiting at distal end 32. Endoscopic tools 36 may include a flexible or rigid endoscopic camera, graspers, forceps, retractors, dissectors, clamps, and so forth, known to those skilled in the art.

Distal end 32 of endoscopic instrument 26, with soft tissue retraction device 20 mounted thereon, is particularly suited for placement within a thoracic cavity 38, containing the lungs and heart, of patient 34. For illustrative purposes, patient 34 is shown in FIG. 3 without a thoracic wall and ribs so as to better visualize the use of soft tissue retraction device within thoracic cavity 38. Expansion element 22 of soft tissue retraction device 20 advantageously functions to urge inflated lung tissue 40 within thoracic cavity 38 away from a target site 42 to form a working space 44 circumscribed by expansion element 22 in which to surgically maneuver endoscopic tools 36. Furthermore, expansion element 22 urges inflated lung tissue 40 away from target site 42 without injuring or destroying lung tissue 40, and without injury or dissection of intervening tissue layers of the chest wall.

Expansion element 22 includes resiliently expandable ribs 46, a first retaining ring 48 adapted to be slidably disposed about distal end 32 of tubular body 28, and a second retaining ring 50 adapted to be fixedly disposed about distal end 32 of tubular body 28. By way of example, second retaining ring 50 is held fixed to distal end 32, by an endcap 52 that prevents expansion element 22 from slipping off of distal end 32 of tubular body 28. Alternatively, second retaining ring 50 may be held fixed to distal end 32 by clips, press-fit elements, O-rings, and so forth.

Each of resiliently expandable ribs 46 includes a first end 54 coupled to first retaining ring 48 and a second end 56 coupled to second retaining ring 50, such that ribs 46 are spaced about a circumference of tubular body 28. Ribs 46 may be formed from surgical steel, medical grade plastics, and the like. Such materials may be sterilized following surgery for subsequent reuse. Ribs 46 are configured to readily bow outwardly, relative to tubular body, in response to an axially applied force (discussed below). Alternatively, ribs 46 may be formed as an inflatable bladder, and force is applied to ribs 46 in the form of a fluid (i.e., air or an isotonic liquid solution) for inflating ribs 46 so that they bow outwardly. Inflatable bladder structures will be described in greater detail hereinbelow.

In an exemplary embodiment, activation mechanism 24 includes tabs 58 placed proximate proximal end 30 of endoscopic instrument 26. For example, a collar structure 60 surrounds tubular body 28 of endoscopic instrument 26 and interconnects tabs 58 and first retaining ring 48. Activation mechanism 24 is used to adjust expansion element 22 from collapsed configuration 21, shown in FIG. 2, to a deployed configuration 62, shown in FIGS. 1 and 3. More specifically, a surgeon may push tabs 58 of activation mechanism 24 toward expansion element 22. When force, as indicated by an arrow 66, is imparted axially on first retaining ring 48 via collar structure 60, first retaining ring 48 is propelled closer to second retaining ring 50. Force 66 subsequently causes ribs 46 to extend outwardly, by a spring-like action, from tubular body 28 to establish working space 44. Thus, ribs 46 form a cage-like structure that circumscribes working space 44.

Collar 60 may then be secured in the forward position so that ribs 46 are held in deployed configuration 62 for the duration of the procedure. The cage-like structure of expansion element 22, makes soft tissue retraction device 20 particularly suited for exploring structures deep within the thoracic cavity, for example, the mediastinum, pericardium, and so forth because ribs 46 can readily urge inflated lung tissue 40 away.

In an alternative configuration, expansion element 22 may be oriented such that the slidable first retaining ring 48 is closer to the tip of distal end 32 of tubular body 28 than the fixed second retaining ring 50. In such a configuration, activation mechanism 24 would be coupled to first retaining ring 48 such that a pulling action on tabs 58 will propel first retaining ring 48 closer to second retaining ring 50, to subsequently cause ribs 46 to extend outwardly from tubular body 28 to establish working space 44.

Soft tissue retraction device 20 may be provided as an after-market device mountable, at the surgeon's discretion, to a variety of endoscopic instruments. For example, an inner diameter of collar structure 60 and first and second retaining rings 48 and 50, respectively, may be manufactured slightly larger than the outer diameter of the tubular body of an endoscopic instrument so that device 20 is simply slid onto the tubular body and secured by a retaining element, such as endcap 52, clip, press-fit element, O-ring, and the like. Alternatively, soft tissue retraction device 20 may be provided affixed to and integral with an endoscopic instrument from an original equipment manufacturer of the endoscopic instrument.

Soft tissue retraction device 20 is advantageously utilized in conjunction with endoscopic instrument 26 during the performance of a video-assisted thoracic surgery (VATS). During such a procedure, patient 34 need not undergo double-lumen endotracheal intubation and single-lung ventilation. Rather, the patient may be sedated and given a local anesthetic at the surgical site. Since intubation, with single-lung ventilation, is not performed, a VATS procedure may be employed for those patients in which a VATS procedure has been previously contraindicated. These patients include critically ill patients unable to tolerate intubation and single-lung ventilation, as well as patients who have previously undergone a pneumonectomy. By performing a VATS procedure on such patients, the pain morbidity, and long recovery duration of the more invasive thoracotomy can advantageously be avoided. In addition, the side effects and serious complications of intubation can advantageously be avoided.

To perform a VATS procedure, once the local anesthetic has taken effect, the surgeon creates an incision, generally less than one inch in diameter, through an intercostal space (i.e., the area between the ribs) of patient 34 into thoracic cavity 38. A portal, known to those skilled in the art, may optionally be inserted into the incision to prevent the incision from closing. Endoscopic instrument 26, with soft tissue retraction device 20 in collapsed configuration 21 mounted thereon, is inserted through the portal and into thoracic cavity 38. After endoscopic instrument 26 is in thoracic cavity 38, the surgeon can then adjust expansion element 22 to deployed configuration 62 via activation mechanism 24 to urge, or push, inflated lung tissue 40 within thoracic cavity 38 away from target site 42. Endoscopic tools 36, extending from distal end 32 of tubular body 28 can then be maneuvered within working space 44 to execute the desired diagnostic or surgical procedure, or to deliver medication to target site 42. Following the procedure, tissue retraction device 20 is adjusted back to collapsed configuration 21 and endoscopic tool 26 is removed from patient 34.

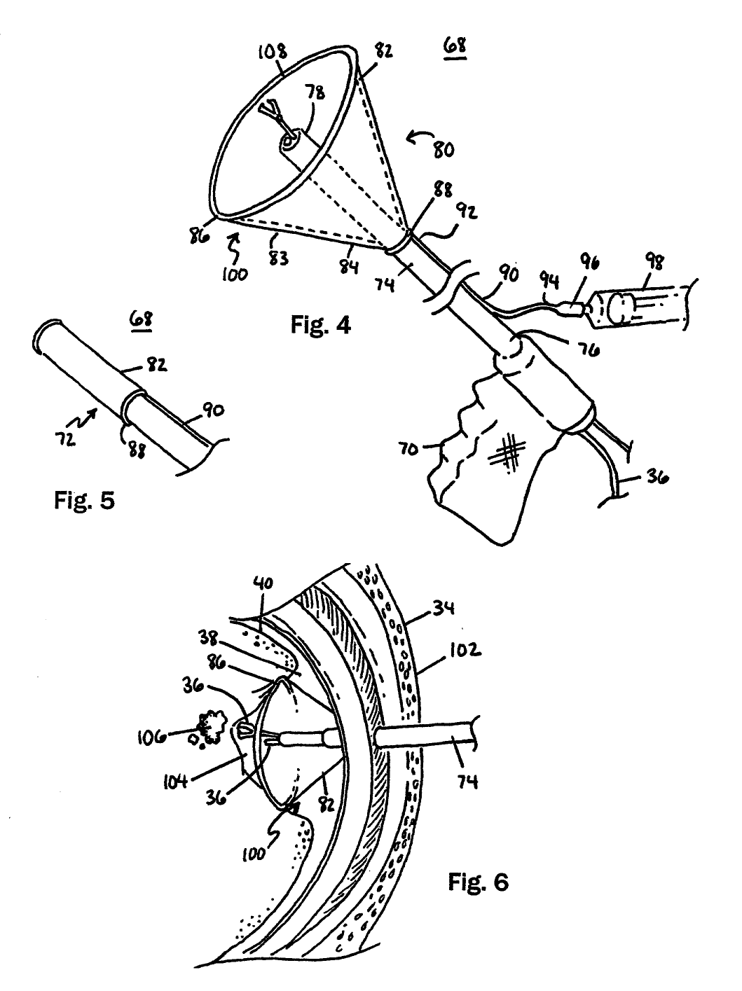

Referring to FIGS. 4-6, FIG. 4 shows a perspective view a soft tissue retraction device 68 mounted on an endoscopic instrument 70 in accordance with an alternative embodiment of the present invention. FIG. 5 shows a perspective view of soft tissue retraction device 68 in a collapsed configuration 72. FIG. 6 shows a cutaway perspective view of soft tissue retraction device 68 in use.

Like endoscopic instrument 26 (FIG. 1), endoscopic instrument 70 includes a tubular body 74 having a proximal end 76 and a distal end 78 adapted for placement in thoracic cavity 38 of patient 34. Endoscopic instrument 70 may include endoscopic tools 36 directed through passages within tubular body 74 from proximal end 76 and exiting at distal end 78.

An expansion element 80 of soft tissue retraction device 68 includes an inflatable bladder 82 configured for mounting on distal end 78 of tubular body 74 of endoscopic instrument 70. Inflatable bladder 82 includes a contoured wall section 83 tapering to a closed vertex 84 and having an open base 86 opposite closed vertex 84. Closed vertex 84 is “closed” by virtue of its attachment to tubular body 74 of endoscopic instrument 70. For example, an O-ring structure 88 is formed at closed vertex 84. O-ring structure 88 is stretched over and held onto tubular body 74 by elastic force. In such a configuration, soft tissue retraction device 68 may be a disposable unit that is optionally placed onto distal end 78 of tubular body 74 prior to insertion into thoracic cavity 38.

Expansion element 80 further includes a fluid passageway 90 having a first end 92 in fluid communication with inflatable bladder 82 and having a second end 94. Fluid passageway 90 is directed toward proximal end 76 of tubular body 74 such that second end 94 is near proximal end 76. An inflation port 96 is coupled to second end 94 of fluid passageway 90. An activation mechanism 98, in the form of a fluid filled syringe, couples to inflation port 96. In a preferred embodiment, fluid within syringe 98 is an isotonic liquid solution, such as saline solution. The saline solution is ejected from syringe 98 and introduced into inflatable bladder 82 via fluid passageway 90. The saline solution subsequently inflates bladder 82 to adjust bladder 82 to a deployed configuration 100, as best shown in FIGS. 4 and 6.

During a VATS procedure, an incision is made through the chest wall 102 of patient 34 at the intercostal region. Endoscopic tool 70 with soft tissue retraction device 68 mounted thereon is inserted through the incision and deployed in thoracic cavity 38. Inflatable bladder 82, in deployed configuration 100, establishes a cone-shaped working space 104 in which open base 86 faces toward distal end 78 of tubular body 74 of endoscopic instrument 70. The establishment of cone-shaped working space 104, with open base 86 facing toward distal end 78, makes inflatable bladder 82 particularly suited for exploring the surface of inflated lung tissue 40. Endoscopic tools 36, such as an endoscopic camera and graspers, may extend from distal end 78 of a tubular body 74 within working space 104 to access a target site 106 on inflated lung tissue 40 for visualization, biopsy, and/or treatment.

In an alternative embodiment, inflatable bladder 82 may additionally include ribs (not shown) built into contoured wall section 83 that provide additional strength to expansion element 80 for noninjuriously urging inflated lung tissue 40 away from target site 106. Alternatively, ribs (not shown) may have first ends coupled to a perimeter 108 of open base 86 and have second ends coupled about tubular body 74 of endoscopic instrument. Thus, the ribs would radiate outwardly from tubular body 74, when soft tissue retraction device 68 is adjusted to deployed configuration 100, to further provide noninjurious retention of inflated lung tissue 40.

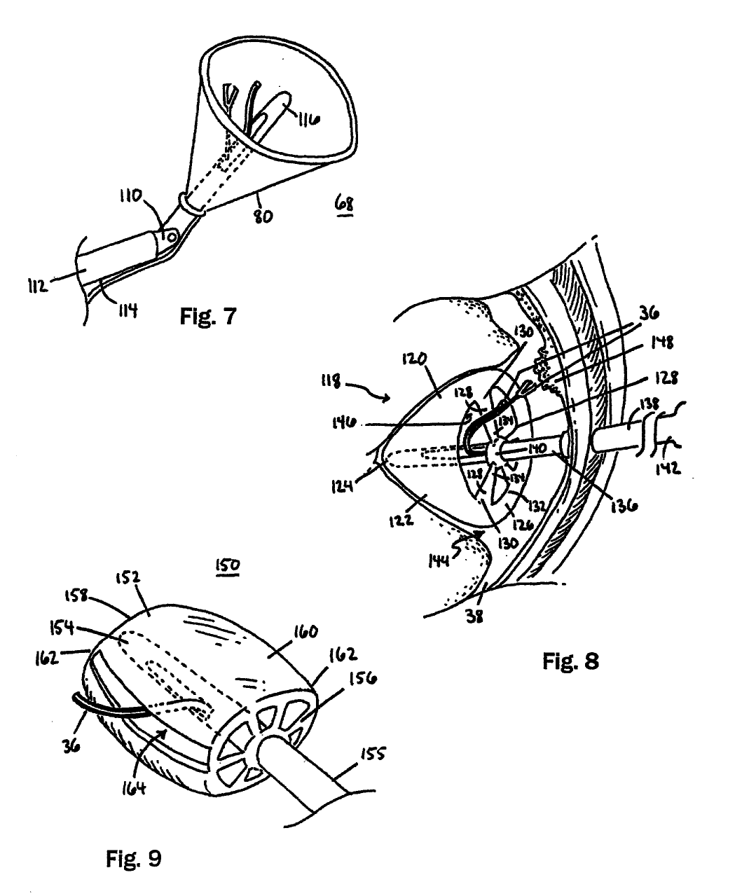

FIG. 7 shows a perspective view of soft tissue retraction device 68 mounted downstream from a moveable joint 110 of a tubular body 112 of another endoscopic instrument 114. Moveable joint 110 is proximate a distal end 116 of tubular body 112. Expansion element 80 of soft tissue retraction device 68 is adapted to be mounted downstream from moveable joint 110. As such, when moveable joint 110 is actuated by a surgeon, distal end 116 of tubular body 112, with expansion element 80 mounted thereon, can be directed toward a target site. Although, soft tissue retraction device 68 is shown mounted downstream from moveable joint 110, other soft tissue retraction devices described herein, such as soft tissue retraction device 20 (FIG. 1), may alternatively be mounted downstream from moveable joint 110.

FIG. 8 shows a cutaway perspective view of another alternative soft tissue retraction device 118 in use. Soft tissue retraction device 118 includes an expansion element 120 having a contoured wall section 122 tapering to a closed vertex 124 and having an open base 126 opposite closed vertex 124. Expansion element 120 may be a resiliently expandable structure in communication with an activation mechanism, such as activation mechanism 24, as discussed in connection with FIGS. 1-3. Alternatively, expansion element 120 may be an inflatable bladder and the activation mechanism may be syringe 98, as discussed in connection with FIGS. 4-6.

Expansion element 120 further includes expandable ribs 128 having first ends 130 coupled to a perimeter 132 of open base 126 and having second ends 134 configured for attachment about a distal end 136 of a tubular body 138 of an endoscopic instrument. By way of example, second ends 134 of ribs 128 may be coupled to an O-ring structure 140. O-ring structure 140 may be stretched over and held onto tubular body 138 by elastic force. In such a configuration, soft tissue retraction device 118 may be optionally placed onto distal end 136 of tubular body 138 prior to insertion into thoracic cavity 38. Alternatively, ribs 128 may be coupled to a slidable ring structure (not shown), with closed vertex 124 attached to a tip of distal end 136. The slidable ring structure is actuated in a manner similar to that discussed in connection with activation mechanism 24 (FIG. 1).

Following insertion into thoracic cavity 38, expansion element 120 is adjusted to a deployed configuration 144, either through a mechanical mechanism or inflation, and ribs 128 radiate outwardly from tubular body 138. In deployed configuration 144, a dome-shaped working space 146 is formed in which open base 126 faces proximal end 142 of tubular body 138, while closed vertex 124 of expansion element 120 noninjuriously urges inflated lung tissue 40 away from a target site 148. The establishment of dome-shaped working space 146, with open base 126 facing toward proximal end 142, makes expansion element 120 particularly suited for exploring, for example, the pleural membrane using endoscopic tools 36.

FIG. 9 shows a perspective view of another alternative soft tissue retraction device 150. Soft tissue retraction device 150 includes an expansion element 152 adapted to be mounted to a distal end 154 of a tubular body 155 of an endoscopic instrument (not shown). Expansion element 152 includes first and second opposing bases 156 and 158, respectively, mounted about tubular body 155. Expansion element 152 further includes a lateral surface 160 interposed between adjoining boundaries 162 of each of first and second bases 156 and 158. Lateral surface 160 is contoured so that expansion element 152 has a generally cylindrical form. Lateral surface 160 includes an opening 164 formed therein for accessing a target site (not shown) within thoracic cavity 38 (FIG. 1). For example, endoscopic tool 36, in the form of an endoscopic camera, can view a target site from opening 164.

Like the structures discussed above, soft tissue retraction device 150 is configured to noninjuriously urge inflated lung tissue 40 (FIG. 3) away from a target site (not shown). Furthermore, soft tissue retraction device can be a resiliently expandable material or an inflatable bladder, and can be permanently affixed to the endoscopic instrument, or can be selectively mounted to the endoscopic instrument.

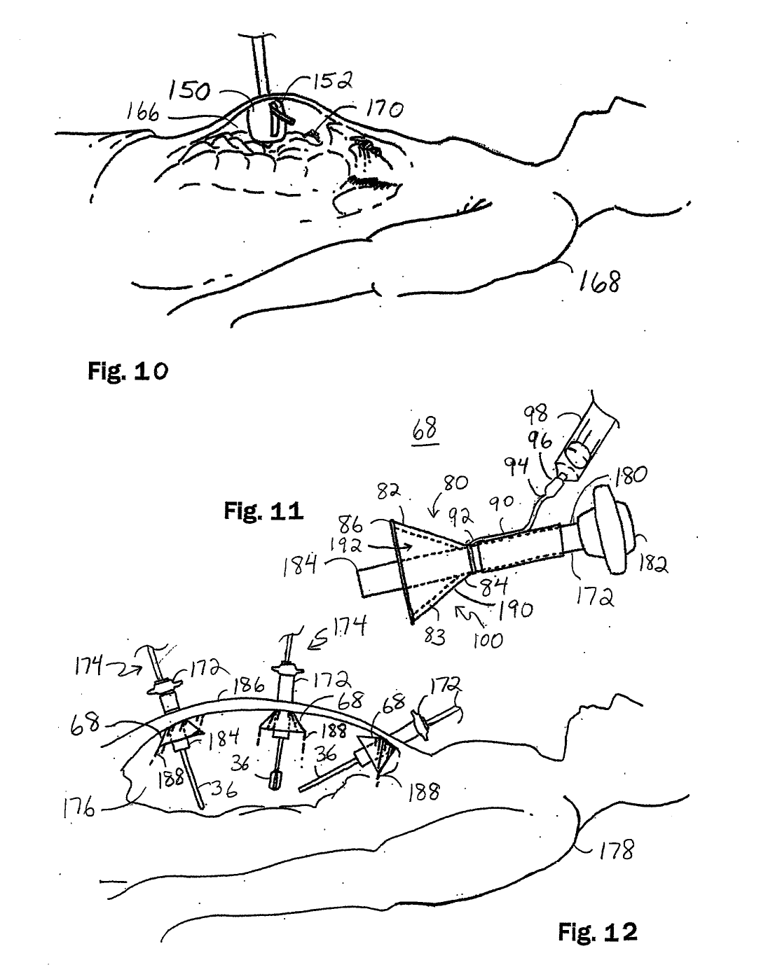

FIG. 10 shows an illustrative perspective view of soft tissue retraction device 150 in use within the abdominal cavity 166 of a patient 168. Although the aforementioned soft tissue retraction devices were described for use within the thoracic cavity of a patient, any of the soft tissue retraction devices, including soft tissue retraction device 150, may alternatively be utilized within abdominal cavity 166 of patient 168. In such a capacity, a surgeon can perform a laparoscopic procedure in which an endoscopic instrument is inserted into abdominal cavity 166 for exploring, diagnosing, and treating disease processes of the internal organs within abdominal cavity 166.

Expansion element 152 of soft tissue retraction device 150 is configured to noninjuriously urge the internal organs of abdominal cavity 166 away from a target site 170. Since expansion element 152 urges the internal organs of abdominal cavity 166 away from target site 170, a surgeon need not distend abdominal cavity 166 with carbon dioxide, as is typically done during a laparoscopic procedure, in order to access the internal organs.

Referring to FIGS. 11-12, FIG. 11 shows a perspective view of soft tissue retraction device 68 mounted on a trocar 172 of an endoscopic instrument 174. FIG. 12 shows an illustrative perspective view of a number of soft tissue retraction devices 68, each being mounted on a separate trocar 172 and in use within the abdominal cavity 176 of a patient 178. Although trocar 172 is described in terms of its use within the abdominal cavity, it should be understood that trocar 172 of endoscopic instrument 174 may alternatively be employed for accessing the thoracic cavity.

Trocar 172 includes a tubular body 180 having a proximal end 182 and a distal end 184 adapted for placement in abdominal cavity 176 of patient 178. Endoscopic instrument 174 may include a number of endoscopic tools 36, directed through one or more passages (not shown) within tubular body 180 from proximal end 182 and exiting at distal end 184.

As discussed previously, expansion element 80 of soft tissue retraction device 68 includes inflatable bladder 82 having contoured wall section 83 tapering to closed vertex 84 and having open base 86 opposite closed vertex 84. Inflatable bladder 82 is configured for mounting on distal end 184 of trocar 172. Thus, closed vertex 84 is “closed” by virtue of its attachment to tubular body 180 of trocar 172. In addition, open base 86 is configured to face toward distal end 184 of trocar 172.

As shown, expansion element 80 further includes fluid passageway 90 having first end 92 in fluid communication with inflatable bladder 82 and having a second end 94. Fluid passageway 90 is directed toward proximal end 182 of tubular body 180 such that second end 94 is near proximal end 182. Activation mechanism 98, in the form of a fluid filled syringe, couples to inflation port 96 at second end 94 of fluid passageway 90 for adjusting bladder 82 to deployed configuration 100.

During a laparoscopic procedure, a surgeon creates one or more incisions through an abdominal wall 186 of patient 178 into abdominal cavity 176. Trocar 172 of endoscopic instrument 174 is inserted, with expansion element 80 in a collapsed configuration, through each of the incisions into abdominal cavity 176. Once distal end 184 of trocar 172 is in abdominal cavity 176, the surgeon adjusts expansion element 80 from the collapsed configuration to deployed configuration 100. Endoscopic tools 36 are then directed through trocar 172 to extend from distal end 184.

With conventional endoscopic instruments, bodily fluid, such as blood, often seeps from the incisions created in the abdominal wall of a patient and visually obstructs the lens of the endoscopic camera device. Such an occurrence undesirably slows down the progress of the laparoscopy because a surgeon is obligated to remove the endoscopic camera from the abdominal cavity to wipe off the lens, apply an anti-fog substance to the lens, and so forth, in order to more clearly view the patient's internal organs on a television monitor.

However, in accordance with the present invention, when expansion element 80 is adjusted to deployed configuration 100, blood 188 is advantageously directed along an outer surface 190 of contoured wall section 83 of expansion element 80 and away from a working space 192 circumscribed by expansion element. Endoscopic tools 36, in particular an endoscopic camera, is then maneuvered within working space 192 so that a surgeon may clearly visualize a target site within abdominal cavity 176.

In summary, the present invention teaches of a soft tissue retraction device for an endoscopic instrument. The soft tissue retraction device, in a number of configurations, includes an expansion element that is adapted to mount on the distal end of a tubular body of the endoscopic instrument. The expansion element pushes against tissue, without injuring the tissue, to form a working space within which endoscopic tools can be surgically maneuvered. The soft tissue retraction device is particularly suited to noninjuriously urge inflated lung tissue away from a target site during a video-assisted thoracic surgery (VATS), thereby eliminating the need for the double-lumen intubation and single-lung ventilation techniques currently in use. In addition, the soft tissue retraction device may be utilized in the abdominal cavity to urge the soft internal organs away from a target site during a laparoscopic procedure. The soft tissue retraction device also serves to direct bodily fluid, such as blood, away from a working space so that a target site may be more clearly visualized. The soft tissue retraction device may be installed as original, sterilizable, equipment on a new endoscopic instrument. Alternatively, it may be provided as an after-market device that readily mounts to a variety of existing and upcoming endoscopic instruments and trocar devices.

Although the preferred embodiments of the invention have been illustrated and described in detail, it will be readily apparent to those skilled in the art that various modifications may be made therein without departing from the spirit of the invention or from the scope of the appended claims.

What is claimed is:

1. A soft tissue retraction device for an endoscopic instrument used to access a body cavity, said endoscopic instrument including a tubular body having a proximal end and a distal end, said distal end being adapted for placement in said body cavity, and said soft tissue retraction device comprising:
an expansion element adapted to mount on said distal end of said tubular body; and
an activation mechanism in communication with said expansion element for adjusting said expansion element from a collapsed configuration to a deployed configuration, said deployed configuration noninjuriously urging tissues within said body cavity away from a target site to form a working space circumscribed by said expansion element in which to surgically maneuver said endoscopic instrument.

2. A soft tissue retraction device as claimed in claim 1 wherein said body cavity is a thoracic cavity, and said deployed configuration of said expansion element is configured to push against inflated lung tissue to access said target site.

3. A soft tissue retraction device as claimed in claim 1 wherein said body cavity is an abdominal cavity, and said deployed configuration of said expansion element is configured to push against internal organs within said abdominal cavity to access said target site.

4. A soft tissue retraction device as claimed in claim 1 wherein said expansion element includes resiliently expandable ribs such that force imparted on said resiliently expandable ribs via said activation mechanism causes said ribs to extend outwardly from said tubular body to establish said working space.

5. A soft tissue retraction device as claimed in claim 4 wherein said expansion element further includes:
a first retaining ring adapted to be slidably disposed about said distal end of said tubular body; and
a second retaining ring adapted to be fixedly disposed about said distal end of said tubular body, and each of said resiliently expandable ribs includes a first end and a second end, said first end being coupled to said first retaining ring, said second end being coupled to said second retaining ring, and force imparted on said first retaining ring via said activation mechanism propels said first retaining ring closer to said second ring to cause said ribs to extend outwardly from said tubular body.

6. A soft tissue retraction device as claimed in claim 1 wherein said expansion element comprises:
an inflatable bladder configured for mounting on said distal end of said tubular body;
a fluid passageway having a first end in fluid communication with said inflatable bladder, said fluid passageway being directed toward said proximal end of said tubular body; and
an inflation port coupled to a second end of said fluid passageway, said activation mechanism coupling to said inflation port for introduction of a fluid from said activation mechanism into said inflatable bladder to adjust said bladder to said deployed configuration.

7. A soft tissue retraction device as claimed in claim 6 wherein said fluid introduced into said inflatable bladder is an isotonic liquid solution.

8. A soft tissue retraction device as claimed in claim 1 wherein said expansion element comprises a contoured wall section tapering to a closed vertex, and having an open base opposite said closed vertex.

9. A soft tissue retraction device as claimed in claim 8 wherein said open base is configured to face toward said proximal end of said tubular body when said expansion element is in said deployed configuration.

10. A soft tissue retraction device as claimed in claim 8 wherein said open base is configured to face toward said distal end of said tubular body when said expansion element is in said deployed configuration.

11. A soft tissue retraction device as claimed in claim 8 wherein said expansion element further comprises expandable ribs having first ends coupled to a perimeter of said open base and having second ends configured for attachment about said tubular body of said endoscopic instrument such that said rib members radiate outwardly from said tubular body in said deployed configuration.

12. A soft tissue retraction device as claimed in claim 1 wherein said expansion element comprises first and second opposing bases and a lateral surface interposed between and adjoining boundaries of each of said bases, an opening being formed in said lateral surface for accessing said target site.

13. A soft tissue retraction device as claimed in claim 1 wherein said tubular body of said endoscopic instrument further comprises a moveable joint proximate said distal end, and said expansion element is configured for mounting downstream from said moveable joint such that actuation of said moveable joint directs said expansion element toward said target site.

14. A soft tissue retraction device for an endoscopic instrument used to access a body cavity, said endoscopic instrument including a tubular body having a proximal end and a distal end, said distal end being adapted for placement in said body cavity, and said soft tissue retraction device comprising:
an expansion element including:
an inflatable bladder adapted for mounting on said distal end of said tubular body, said inflatable bladder having a contoured wall section tapering to a closed vertex, and having an open base opposite said closed vertex;
a fluid passageway having a first end in fluid communication with said inflatable bladder, said fluid passageway being directed toward said proximal end of said tubular body; and
an inflation port coupled to a second end of said fluid passageway; and
an activation mechanism coupling to said inflation port for introducing a fluid from said activation mechanism into said inflatable bladder to adjust said bladder from a collapsed configuration to a deployed configuration, said deployed configuration noninjuriously urging tissues within said body cavity away from a target site to form a working space circumscribed by said expansion element in which to surgically maneuver said endoscopic instrument.

15. A soft tissue retraction device as claimed in claim 14 wherein said open base is configured to face toward said proximal end of said tubular body when said expansion element is in said deployed configuration.

16. A soft tissue retraction device as claimed in claim 14 wherein said open base is configured to face toward said distal end of said tubular body when said expansion element is in said deployed configuration.

17. A soft tissue retraction device as claimed in claim 14 wherein said expansion element further includes rib members having first ends coupled to a perimeter of said open base and having second ends configured for attachment about said tubular body of said endoscopic instrument such that said rib members radiate outwardly from said tubular body in said deployed configuration.

18. A soft tissue retraction device as claimed in claim 14 wherein said body cavity is a thoracic cavity, and said deployed configuration of said expansion element is configured to push against inflated lung tissue to access said target site.

19. An endoscopic instrument used to access a thoracic cavity comprising:
a tubular body having a proximal end and a distal end, said distal end being adapted for placement in said body cavity, and said distal end having an endoscopic tool extending therefrom; and
a soft tissue retraction device having an expansion element mounted on said distal end of said tubular body and having an activation mechanism located near said proximal end of said tubular body, said activation mechanism being in communication with said expansion element for adjusting said expansion element from a collapsed configuration to a deployed configuration, said deployed configuration noninjuriously pushing inflated lung tissue within said chest cavity away from a target site to form a working space circumscribed by said expansion element in which to surgically maneuver said endoscopic tool.

20. An endoscopic instrument as claimed in claim 19 wherein said expansion element includes expandable ribs arranged about said tubular body such that force imparted on said expandable ribs via said activation mechanism causes said ribs to extend outwardly from said tubular body to establish said working space.

21. An endoscopic instrument as claimed in claim 20 wherein said expansion element further includes:
a first retaining ring slidably disposed about said distal end of said tubular body; and
a second retaining ring fixedly disposed about said distal end of said tubular body, and each of said expandable ribs includes a first end and a second end, said first end being coupled to said first retaining ring, said second end being coupled to said second retaining ring, and force imparted on said first retaining ring via said activation mechanism propels said first retaining ring closer to said second ring to cause said ribs to extend outwardly from said tubular body.

22. An endoscopic instrument as claimed in claim 20 wherein said expansion element further includes a contoured wall section tapering to a closed vertex, and having an open base opposite said closed vertex, said ribs having first ends coupled to a perimeter of said open base and having second ends configured for attachment about said tubular body.

23. A method of performing video-assisted thoracic surgery (VATS) at a target site in a thoracic cavity of a body in the presence of inflated lung tissue, said method comprising:
creating an incision through an intercostal space of said body into said thoracic cavity;
inserting a endoscopic instrument, with a soft tissue retraction device mounted thereon, through said incision into said thoracic cavity, said endoscopic instrument including a tubular body having a proximal end and a distal end, said distal end being adapted for placement in said body cavity, and said distal end having an endoscopic tool extending therefrom, said soft tissue retraction device comprising an expansion element mounted on said distal end of said tubular body and an activation mechanism in communication with said expansion element for adjusting said expansion element from a collapsed configuration to a deployed configuration, said inserting operation being performed with said expansion element in said collapsed configuration;
adjusting said expansion element to said deployed configuration via said activation mechanism to noninjuriously urge said inflated lung tissue within said thoracic cavity away from a target site to form a working space circumscribed by said expansion element; and
maneuvering said endoscopic tool within said working space to access said target site.

24. A method as claimed in claim 23 wherein said expansion element includes resiliently expandable ribs, a first retaining ring slidably disposed about said distal end of said tubular body, and a second retaining ring fixedly disposed about said distal end of said tubular body, and each of said resiliently expandable ribs includes a first end and a second end, said first end being coupled to said first retaining ring, said second end being coupled to said second retaining ring, and said adjusting operation comprises imparting force on said first retaining ring via said activation mechanism to propel said first retaining ring closer to said second ring to cause said ribs to extend outwardly from said tubular body to establish said deployed configuration.

25. A method as claimed in claim 23 wherein said expansion element includes an inflatable bladder mounted on said distal end of said tubular body, a fluid passageway having a first end in fluid communication with said inflatable bladder, said fluid passageway being directed toward said proximal end of said tubular body, and an inflation port coupled to a second end of said fluid passageway, and said adjusting operation comprises:
coupling said activation mechanism to said inflation port; and
introducing an isotonic liquid solution from said activation mechanism into said inflatable bladder to adjust said bladder to said deployed configuration.

26. A method as claimed in claim 23 wherein said tubular body of said endoscopic instrument further includes a moveable joint proximate said distal end, said expansion element is mounted downstream from said moveable joint, and said method further comprises actuating said moveable joint to direct said expansion element toward said target site.

27. A method of visualizing a target site in a body cavity in the presence of visually obstructive bodily fluid, said method comprising:
creating an incision through a wall of said body into said body cavity;
inserting a trocar of an endoscopic instrument through said incision into said body cavity, said trocar having a soft tissue retraction device mounted thereon, said trocar including a tubular body having a proximal end and a distal end, said distal end being adapted for placement in said body cavity, and said soft tissue retraction device including an expansion element mounted on said distal end of said tubular body and an activation mechanism in communication with said expansion element for adjusting said expansion element from a collapsed configuration to a deployed configuration, said inserting operation being performed with said expansion element in said collapsed configuration;
adjusting said expansion element to said deployed configuration via said activation mechanism to direct said visually obstructive bodily fluid away from a target site and form a working space circumscribed by said expansion element;
directing an endoscopic tool through said trocar such that said endoscopic tool extends from said distal end; and
maneuvering said endoscopic tool within said working space to visualize said target site.

28. A method as claimed in claim 27 wherein said expansion element comprises a contoured wall section tapering to a closed vertex, and having an open base opposite said closed vertex, said open base being configured to face toward said distal end of said tubular body when said expansion element is in said deployed configuration, and said adjusting operation causes said obstructive bodily fluid to be directed along an outer surface of said contoured wall section and away from said working space.

29. A method as claimed in claim 27 wherein said obstructive bodily fluid is blood.
